(19) United States
(12) Patent Application Publication
(10) Pub. No.: US 2022/0288324 A1
(43) Pub. Date: Sep. 15, 2022

(54) DRUG DELIVERY DEVICE

(71) Applicant: AMGEN INC., Thousand Oaks, CA (US)

(72) Inventors: Austin Davis, Thousand Oaks, CA (US); Lars Eilertsen, Søborg (DK); David Lavmand Muller, Thousand Oaks, CA (US); Rasmus Ohlenschlaeger, Hellerup (DK); Gregory Thomas Nowak, Somerville, MA (US); Kasper Poder, Tikøb (DK); Dylan Bourelle, Woodland Hills, CA (US)

(21) Appl. No.: 17/690,038

(22) Filed: Mar. 9, 2022

Related U.S. Application Data
(60) Provisional application No. 63/159,335, filed on Mar. 10, 2021.

(51) Int. Cl. A61M 5/32

(52) U.S. Cl. CPC A61M 5/3202

(57) ABSTRACT

A drug delivery device including a housing, a drug storage container, and a removable cap is provided. The housing includes a housing camming feature and a longitudinal axis and includes an opening. The drug storage container includes a delivery member having an insertion end configured to extend at least partially through the opening during a delivery state. The removable cap defines a cap camming feature and is configured to be removably coupled with the housing such that the removable cap has a storage position where the removable cap is coupled with the housing and at least partially covering the opening and a removed position where the removable cap is not coupled with the housing. The cap camming feature and the housing camming feature are configured to translate rotational motion into axial motion such that, upon rotational movement of the removable cap, the cap camming feature and/or the housing camming feature urge the removable cap along the longitudinal axis. The housing camming feature and the cap camming feature are each visible to a user of the drug delivery device to signal the camming function of the housing camming feature and the cap camming feature.

### FIELD OF DISCLOSURE

The present disclosure relates to drug delivery devices and, more particularly, devices for automatically injecting a drug into a patient.

### BACKGROUND

A general aversion to exposed needles, as well as health and safety issues, have led to the development of drug delivery devices which conceal a needle or other insertion member prior to use and which automate various aspects of an injection process. Such devices offer a variety of benefits as compared with traditional forms of drug delivery including, for example, delivery via a conventional syringe.

A drug delivery device may incorporate various mechanisms to implement various automated features. Such features may include automatically covering a needle in a pre-delivery and/or post-delivery state, automatically inserting a needle and/or a cannula into a user, automatically activating a drive mechanism, automatically indicating to the user that drug delivery is complete, among other features. The device may also include an additional needle-covering feature such as a removable cap that utilized when the device is in a storage state. The removable cap may be manually removed by the user, which may require a removal force of a minimum magnitude and/or in a particular direction.

Additionally, some users may not be familiar with all the features or functions of drug delivery devices. For example, some users may not readily appreciate that the cap should be removed before use or how the cap can/should be removed.

The present disclosure sets forth drug delivery devices embodying advantageous alternatives to existing drug delivery devices, and removable cap removal features, and that may address one or more of the challenges or needs mentioned herein.

### SUMMARY

One aspect of the present disclosure provides a drug delivery device including a housing, a drug storage container, and a removable cap. The housing may include a housing camming feature and a longitudinal axis and includes an opening. The drug storage container may include a delivery member having an insertion end configured to extend at least partially through the opening during a delivery state. The removable cap may define a cap camming feature and is configured to be removably coupled with the housing such that the removable cap has a storage position where the removable cap is coupled with the housing and at least partially covering the opening and a removed position where the removable cap is not coupled with the housing. The cap camming feature and the housing camming feature are configured to translate rotational motion into axial motion such that, upon rotational movement of the removable cap, the cap camming feature and/or the housing camming feature urge the removable cap along the longitudinal axis. The housing camming feature and the cap camming feature are each visible to a user of the drug delivery device to signal the camming function of the housing camming feature and/or the cap camming feature.

The cap camming feature may define a wave shape. The removable cap may include a generally cylindrical body portion defining an annular leading rim and an end wall generally perpendicular to the body portion and wherein the annular leading rim defines the wave shape. The annular leading rim may define two wave shapes.

The housing may define a generally cylindrical outer surface and the housing camming feature may include a protrusion extending away from a generally cylindrical outer surface. The protrusion may be aligned with the wave shape of the cap camming feature when the removable cap is in the storage position. The protrusion may abut the wave shape of the cap camming feature when the removable cap is in the storage position. The protrusion may define a wave surface corresponding to the wave shape of the cap camming feature.

The housing may include two protrusions, each extending away from a generally cylindrical outer surface. The housing camming feature and the cap camming feature may each be defined by or positioned on an outer surface of the drug delivery device.

The housing camming feature and/or the cap camming feature may include an aspect to improve the visibility thereof. For example, the aspect to improve the visibility may include a bright color.

The removable cap may include at least one rotation-assistance feature, such as a fin.

Another aspect of the present disclosure provides a drug delivery device including a housing, a drug storage container, and a removable cap. The housing may include a housing camming feature and a longitudinal axis and includes an opening. The drug storage container may include a delivery member having an insertion end configured to extend at least partially through the opening during a delivery state. The removable cap may define a cap camming feature and is configured to be removably coupled with the housing such that the removable cap has a storage position where the removable cap is coupled with the housing and at least partially covering the opening and a removed position where the removable cap is not coupled with the housing. The cap camming feature and the housing camming feature are configured to translate rotational motion into axial motion such that, upon rotational movement of the removable cap, the cap camming feature and/or the housing camming feature urge the removable cap along the longitudinal axis. The housing camming feature and the cap camming feature may each be defined by or positioned on an outer surface of the drug delivery device to signal the camming function of the housing camming feature and/or the cap camming feature.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a perspective view of an exemplary drug delivery device in accordance with various embodiments, with the device removable cap present and coupled with the housing;

FIG. 2 is a front view of the drug delivery device in FIG. 1;

FIG. 3A is a perspective view of a distal portion of the drug delivery device in FIG. 1, with the removable cap in the process of being removed therefrom;

FIG. 3B is a perspective view similar to FIG. 3A, with the removable cap rotated more than in FIG. 3A to further decouple the cap from the housing;

FIG. 4 is a perspective view of a distal portion of the drug delivery device in FIG. 1, with the removable cap removed therefrom;

FIG. 5 is a cross-sectional view of a portion of the drug delivery device in FIG. 1, with the removable cap present and coupled with the housing;

FIG. 6 is a front view of the housing of another exemplary drug delivery device in accordance with various embodiments;

FIG. 7 is a side view of the drug delivery device housing in FIG. 6;

FIG. 8 is a perspective view of the distal portion of the housing in FIG. 6 positioned adjacent to a removable cap configured to be removably coupled with the housing;

FIG. 9 is a perspective view of a distal portion of another exemplary drug delivery device in accordance with various embodiments, with the device removable cap present and coupled with the housing;

FIG. 10 is a perspective view a distal portion of yet another exemplary drug delivery device in accordance with various embodiments, with the device removable cap present and coupled with the housing;

FIG. 11 is a perspective view a distal portion of yet another exemplary drug delivery device in accordance with various embodiments, with the device removable cap present and coupled with the housing;

FIG. 12 is a perspective view a distal portion of yet another exemplary drug delivery device in accordance with various embodiments, with the device removable cap present and coupled with the housing;

FIG. 13 is a perspective view a distal portion of yet another exemplary drug delivery device in accordance with various embodiments, with the device removable cap present and coupled with the housing; and

FIG. 14 is a perspective view a distal portion of a removable cap of yet another exemplary drug delivery device in accordance with various embodiments.

### DETAILED DESCRIPTION

The present disclosure generally relates to drug delivery devices operable by a user for administering a drug, or in the case where a patient is the user, self-administering a drug. The device includes a housing with a housing camming feature and a longitudinal axis and includes an opening. The device also includes a removable cap with a cap camming feature and is configured to be removably coupled with the housing such that the removable cap has a storage position where the removable cap is coupled with the housing and at least partially covering the opening and a removed position where the removable cap is not coupled with the housing. The cap camming feature and the housing camming feature are configured to translate rotational motion into axial motion such that, upon rotational movement of the removable cap, the cap camming feature and/or the housing camming feature urge the removable cap along the longitudinal axis. The housing camming feature and the cap camming feature are each visible to a user of the drug delivery device to signal the camming function of the housing camming feature and the cap camming feature

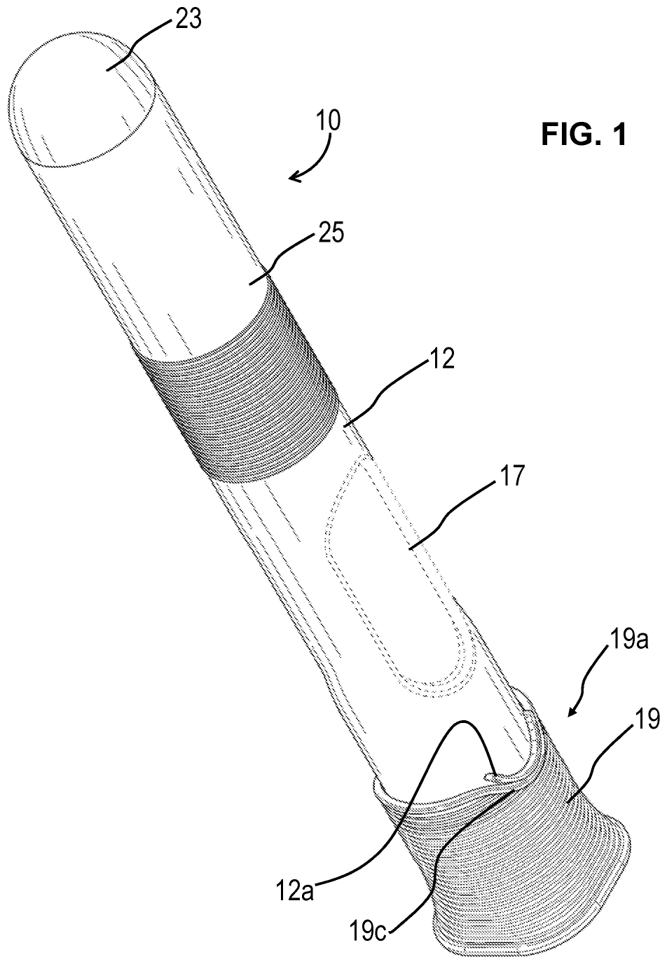

FIGS. 1-5 illustrate several views of an embodiment of a drug delivery device 10 for delivering a drug, which may also be referred to herein as a medicament or drug product. The drug may be, but is not limited to, various biologicals such as peptides, peptibodies, or antibodies. The drug may be in a fluid or liquid form, although the disclosure is not limited to a particular state.

Various implementations and configurations of the drug delivery device 10 are possible. The present embodiment of the drug delivery device 10 is configured as a single-use, disposable injector. In other embodiments, the drug delivery device 10 may be configured as multiple-use reusable injector. The drug delivery device 10 is operable for self-administration by a patient or for administration by caregiver or a formally trained healthcare provider (e.g., a doctor or nurse). The exemplary the drug delivery devices shown in the figures may take the form of an autoinjector or pen-type injector, and, as such, may be held in the hand of the user over the duration of drug delivery, but may also or alternatively be suitable for other drug delivery devices and/or configurations.

The configuration of various components included in the drug delivery device 10 may depend on the operational state of the drug delivery device 10. The drug delivery device 10 may have a storage state, a pre-delivery state, a delivery or dosing state, and a post-delivery state, although fewer or more states are also possible. For example, each state may have several sub-states or stages. The storage state may correspond to the configuration of the drug delivery device 10 in FIGS. 1-2 and 5, where the delivery device includes a removable cap in a storage position. In some embodiments, the storage state may exist in the time between when the drug delivery device 10 leaves a manufacturing facility and when a patient or user removes the cap. The pre-delivery stage may correspond to the configuration of the drug delivery device 10 after the removable cap has been removed but prior to activation of the device by the user. This may include the moments in time after the user has removed the removable cap, while the user is first positioning the drug delivery device 10 against the injection site, but before dosing has begun. The delivery state may correspond to the configuration of the drug delivery device 10 while drug delivery, also referred to herein as dosing, is in progress. The post-delivery state may correspond to the configuration of the drug delivery device 10 after drug delivery is complete and/or when a stopper is arranged in an end-of-dose position in a drug storage container.

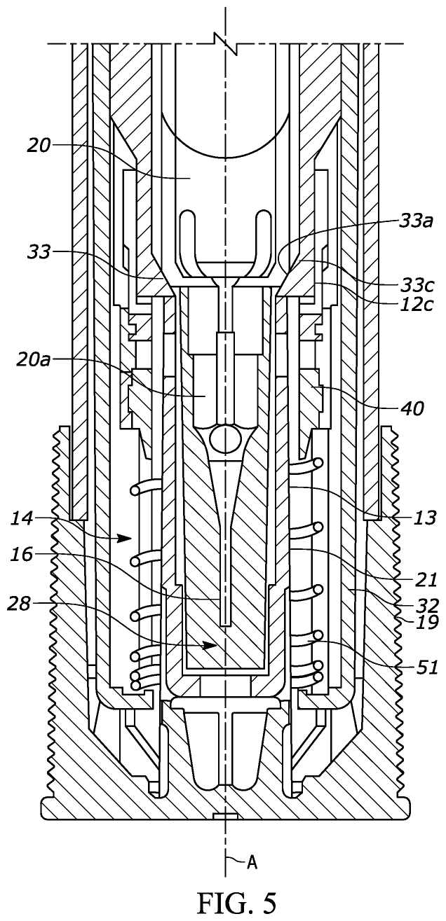

As shown in FIGS. 1-5, the drug delivery device 10 includes an outer casing or housing 12. In some embodiments, the housing 12 may be sized and dimensioned to enable a person to grasp the injector 10 in a single hand. The housing 12 may have a generally elongate shape, such as a cylindrical shape, and extend along a longitudinal axis A between a proximal end and a distal end. An opening 14 (FIG. 5) may be formed in the distal end to permit an insertion end 28 of a delivery member 16 to extend outside of the housing 12. A transparent or semi-transparent inspection window 17 may be positioned in a wall of the housing 12 to permit a user to view component(s) inside the drug delivery device 10, including a drug storage container 20. Viewing the drug storage container 20 through the window 17 may allow a user to confirm that drug delivery is in progress and/or complete. A removable cap 19 may cover the opening 14 at the distal end of the device prior to use of the drug delivery device 10, and, in some embodiments, may including a gripper 13 (FIG. 5) configured to assist with removing a sterile barrier 21 (e.g., a rigid needle shield (RNS), a non-rigid needle shield (nRNS), etc.) mounted on the insertion end 28 of the delivery member 16. The gripper 13 may include one or more inwardly protruding barbs or arms that frictionally or otherwise mechanically engage the sterile barrier 21 to pull the sterile barrier 21 with the removable cap 19 when the user separates the removable cap 19 from the housing 12. Thus, removing the removable cap 19 has the effect of removing the sterile barrier 21 from the delivery member 16.

The device may include a drive mechanism that is configured to store energy and, upon or in response to activation of the drive mechanism by the user, release or output that energy to drive a plunger to expel a drug from the drug storage container 20 through the delivery member 16 into the patient.

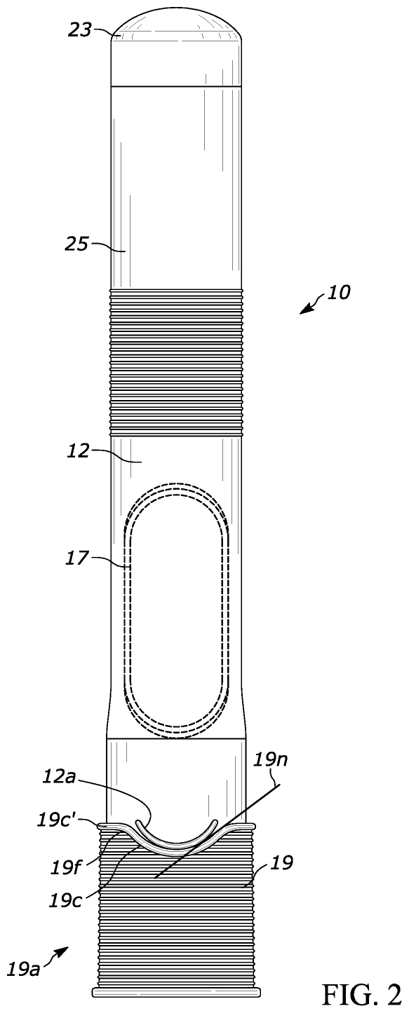

As best shown in FIGS. 1-2, in one embodiment the housing 12 may include two separate and interconnected structures: a rear end cap 23 (e.g., a rear cover) at the proximal end of the drug delivery device 10; and a tubular housing 25 extending substantially completely along the length of the drug delivery device 10 and defining the opening 14. Additionally or alternatively, the housing 12 may include fewer or more components, such as a two-piece tubular housing having front and rear portions. The tubular housing 25 may have a hollow and generally cylindrical or tubular shape, and the rear end cap 23 may have a generally hemispherical shape or a hollow cylindrical shape with an open end and a closed off end. In some embodiments, the rear end cap 23 and the tubular housing 25, and any components to be positioned therein, may be assembled together to define different sub-assemblies. In alternative embodiments, the housing 12 may be constructed in one piece, such that the housing 12 is defined by a single, monolithic structure that integrates a rear cap and tubular housing in a single component.

The drug storage container 20 is disposed within an interior space of the housing 12 and is configured to contain a drug. The drug storage container 20 may be pre-filled and shipped, e.g., by a manufacturer, to a location where the drug storage container 20 is combined with a remainder of the drug delivery device 10. For example, the drug 22 may be distributed and/or provided to patients in more than one use case, such as a as a pre-filled syringe or as an autoinjector including a pre-filled syringe. By utilizing the same or similar syringe components in either case, at least some of above steps such as filling, labeling, packaging, shipping, and distribution may be streamlined or simplified for two different use cases. As another example, in the event that multiple use cases utilize some or all of the same syringe components, some regulatory pathways to marketing and/or distributing the drug may be streamlined and/or simplified for at least one of the multiple use cases.

In some embodiments, a volume of the drug 22 included in the reservoir of the drug storage container 20 may be equal to 1 mL, or equal to approximately (e.g., ±10%) 1 mL, or equal to 2.5 mL, or equal to approximately (e.g., ±10%) 2.5 mL, or equal to 3 mL, or equal to approximately (e.g., ±10%) 3 mL, or less than or equal to approximately (e.g., ±10%) 1 mL, or less than or equal to approximately (e.g., ±10%) 2 mL, or less than or equal to approximately (e.g., ±10%) 3 mL, or less than or equal to approximately (e.g., ±10%) 4 mL, or less than approximately (e.g., ±10%) 5 mL, or less than or equal to approximately (e.g., ±10%) 10 mL, or within a range between approximately (e.g., ±10%) 1-10 mL, or within a range between approximately (e.g., ±10%) 1-5 mL, or within a range between approximately (e.g., ±10%) 1-4 mL, or within a range between approximately (e.g., ±10%) 1-3 mL, or within a range between approximately (e.g., ±10%) 1-2.5 mL.

The delivery member 16 is connected or operable to be connected in fluid communication with the reservoir of the drug storage container 20. A distal end of the delivery member 16 may define the insertion end 28 of the delivery member 16. The insertion end 28 may include a sharpened tip of other pointed geometry allowing the insertion end 28 to pierce the patient's skin 5 and subcutaneous tissue during insertion of the delivery member 16. The delivery member 16 may be hollow and have an interior passageway. One or more openings may be formed in the insertion end 28 to allow drug to flow out of the delivery member 16 into the patient.

In one embodiment, the drug storage container 20 may be a pre-filled syringe and has a staked, hollow metal needle for the delivery member 16. Here, the needle is fixed relative to the wall of the drug storage container 20 and may be in permanent fluid communication with the reservoir of the drug storage container 20. In other embodiments, the needle may be coupled to the drug storage container 20 via a Luer Lock or other suitable connection. In yet other embodiments, the drug storage container 20 may be a needle-less cartridge, and, as such, initially may not be in fluid communication with the delivery member 16. In such embodiments, the drug storage container 20 may move toward a proximal end of the delivery member 16, or vice versa, during operation of the drug delivery device 10 such that the proximal end of the delivery member 16 penetrates through a septum covering an opening in the drug storage container 20 thereby establishing fluid communication between the reservoir of the drug storage container 20 and the delivery member 16.

The drug storage container 20 may include a body portion with a distal end 20a and a proximal end (not shown). The drug storage container 20 may be fixed relative to the housing 12 such that the drug storage container 20 does not move relative to the housing 12 once installed in the housing 12. As such, the insertion end 28 of the delivery member 16 extends permanently through the opening 14 in the housing 12 in the pre-delivery, delivery, and post-delivery states. For example, as shown in FIG. 2, the delivery member 16 extends beyond a distal end of the housing 12 that defines the opening 14. However, in some configurations, such as the storage configuration shown in FIG. 2, the delivery member 16 is covered/protected by the sterile barrier 21 and a guard member 32 that surrounds the delivery member 16 and protects against or reduces the likelihood of unintended or premature needle stick.

The device may also include a container holder 33 configured to secure the drug storage container 20 with respect to the housing 12, such as by preventing distal movement of the drug storage container 20 during actuation of the plunger. The container holder 33 may include a plurality of flanges 33c that each include an arcuate, sloped surface 33a that substantially matches the arcuate shape of a shoulder portion of the drug storage container 20. As a more specific example, when the drug storage container 20 is inserted within the container holder 33, the flanges 33c cooperate to support the shoulder portion and limit the travel of the drug storage container 20 in the distal direction. The housing 12 may includes a plurality of lock slots 12c that each receive respective flanges 33c of the container holder 33 to prevent and/or restrict relative movement between the respective components 12, 33. As a result, when fully assembled the storage container 20, the container holder 33, and the housing 12 are all substantially or completely fixed with respect to each other.

The device may also include a lock ring 40 configured to lock the guard member 32 in the extended position once the device has reached a certain state, such as the injection or the post-injection state. The lock ring 40 shown in FIG. 5 is centered and rotates about the longitudinal axis A. In some embodiments, the lock ring biasing member 51 may include a compression spring (e.g., a helical compression spring). The lock ring 40 may also serve to provide an initial resistance to movement of the guard member 32. For example, the initial resistance may be configured to facilitate insertion of the delivery member 16 into the patient by utilizing, harness, or otherwise taking advantage of inertial forces. In other words, the lock ring 40 and/or other components may provide an initial resistance to movement of the guard member 32 to build-up the user inputted force.

These and other aspects of operation of an exemplary drug delivery device are discussed in more detail in U.S. application Ser. No. 17/035,851, filed Sep. 29, 2020, the entire contents of which are incorporated by reference.

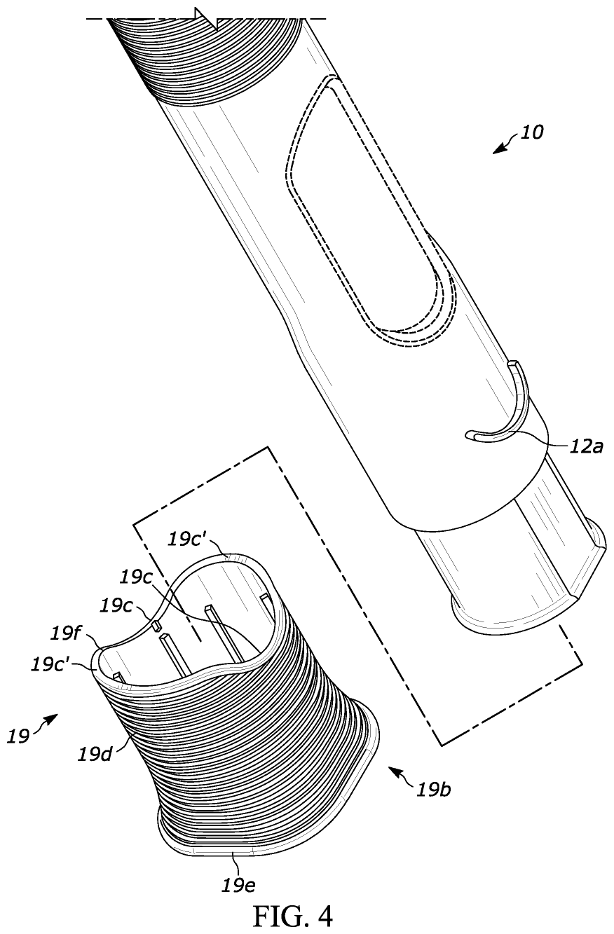

As discussed above, the removable cap 19 may have a storage position (FIGS. 1, 2, 5) where the removable cap 19 is coupled with the housing 12 and a removed position (FIG. 4) where the removable cap 19 is removed from and not coupled with the housing 12. As also discussed above, the device 10 may include a sterile barrier 21 that is removed from the delivery member 16 when the removable cap is removed from the housing 12. The sterile barrier 21 may have a relatively snug or relatively high-friction fit with the drug storage container 20 to maintain the sterility of the delivery member 16 and/or to prevent air from entering the drug storage container 20. For example, in order to reduce the likelihood of contamination and/or occlusions or evaporated drug, it may be desirable to prevent or reduce the likelihood of air entering the drug storage container and/or the delivery member 16. Additionally or alternatively, it may be desirable to have a relatively snug or relatively high-friction fit between the sterile barrier 21 and the drug storage container 20 to prevent or reduce the likelihood of inadvertent needle sticks. For these or other reasons, it may also or alternatively be desirable to have a relatively snug or relatively high-friction fit between the removable cap 19 and the housing 12. The sterile barrier 21 and the removable cap 19 may also be coupled with their respective components (e.g., drug storage container 20 and housing 12) via other suitable features, such as coupling tab/slot connections, breakable connections such as perforated seals, threaded connections, or other features that achieve relatively secure but removable connections between respective components.

As a result of these coupling forces, features, and/or other factors, some device users may experience difficulty or discomfort removing the removable cap 19. As an example, some device users may have difficulty removing the cap 19 via axial forces alone (along axis A). In other words, some device users may have difficulty in pulling the cap 19 off of/away from the housing 12. The cap 19 shown in FIGS. 1-5 includes a plurality of ribs to help the user grip the surface of the cap when removing the same.

The device 10 shown in FIGS. 1-5 also includes camming features to translate rotational motion into axial motion such that, upon rotational movement of the removable cap 19, the removable cap 19 is urged away from the housing 12, thereby facilitating and/or easing removal of the cap 19. For example, the housing 12 includes a housing camming feature 12a and a cap camming feature 19c. As a more specific example, to remove the cap 19 from the housing 12 via axial force/movement only (e.g., “straight-pull force”), a user may be required to exert 45 Newtons or less; approximately 40 to 45 Newtons; approximately 35 to 40 Newtons; approximately 30 to 35 Newtons; approximately 25 to 30 Newtons; approximately 20 to 25 Newtons; approximately 15 to 20 Newtons; approximately 10 to 15 Newtons; approximately 5 to 10 Newtons; or less than approximately 5 Newtons. In the device 10 shown in FIGS. 1-5, removing the cap 19 requires approximately 10 to 15 Newtons of straight-pull force.

The cap camming feature 19c shown in FIGS. 1-5 defines a wave shape, such as an arc-shaped surface. As a more specific example, the removable cap 19 shown in the figures includes a generally cylindrical body portion 19d and an end wall 19e that is generally perpendicular to the body portion 19d at the distal end of the cap 19. The body portion 19d defines a generally annular leading rim 19f at the proximal end of the cap 19. The leading rim 19f defines the wave shaped cap camming feature 19c. As an even more specific example, the leading rim 19f shown in the figures defines two wave shaped camming surfaces 19c and two relatively flat surfaces 19c′ that extend between wave shaped camming surfaces 19c. In other words, the two wave shaped camming surfaces 19c and the two relatively flat surfaces 19c′ cooperate to define the leading rim 19f. Alternatively, the leading rim 19f may define a continuous wave shape such as a continuous sinusoidal wave or another continuous wave shape. For the purposes of this application, the term “continuous” should be interpreted to mean that the wave shape continues around the entire perimeter of the leading edge rather than alternating wave shaped and flat surfaces.

The housing camming feature 12a shown in FIGS. 1-5 defines a wave shape, such as an arc-shaped protrusion extending away from the outer surface 25 of the housing 12. As a more specific example, the housing camming feature 12a is a protrusion having a shape that is not unlike a “smile” or a “crescent moon” shape. As an even more specific example, the housing 12 shown in the figures defines two wave shaped camming features 12a.

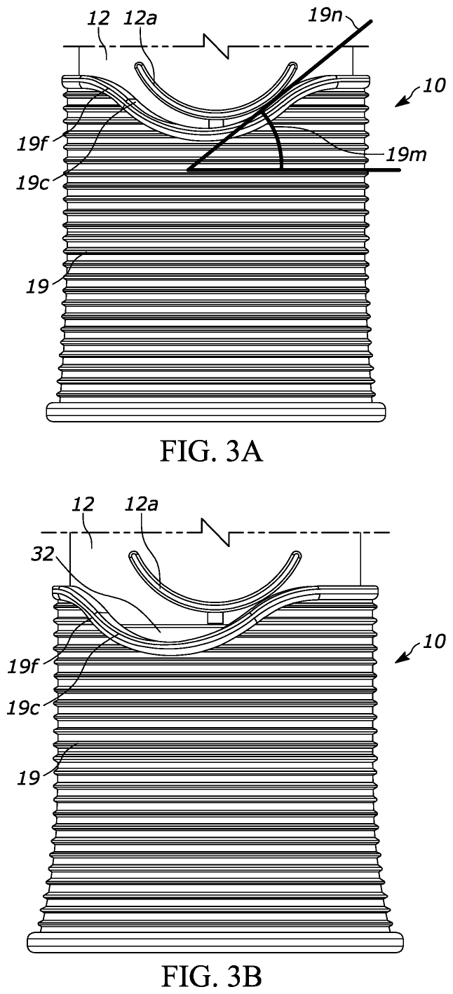

When the removable cap 19 is in the storage position 19a shown in FIGS. 1, 2, 5, the cap camming features 19c engage or abut the housing camming features 12a. Additionally, the respective camming features 12a, 19c shown in the figures have matching or mirrored shapes such that the respective surfaces 12a, 19c slide smoothly/easily across each other. For example, when the removable cap 19 is rotated (either clockwise or counterclockwise) with respect to the housing 12, the housing camming features 12a, 19c rotate with respect to each other and urge the removable cap 19 away from the housing 12 along axis A. In other words, the camming features 12a, 19c translate rotational motion into axial motion to remove or assist with removal of the cap 19. As a more specific example, FIG. 3A shows the distal portion of the device 10 after the removable cap 19 has been rotated with respect to the housing 12, thereby urging the removable cap 19 in the distal direction and away from the housing 12. The rotation shown in FIG. 3A is a relatively small rotation (5-10 degrees around axis A) but may still be sufficient to overcome at least the initial coupling forces between the removable cap 19 and the housing 12, on one hand, and the sterile barrier 21 and the drug storage container 20, on the other hand. As a result, even a relatively small rotation may facilitate and/or ease removal of the cap 19. FIG. 3B shows the distal portion of the device 10 after the removable cap 19 has been rotated farther, thereby urging the removable cap 19 farther in the distal direction and away from the housing 12. The rotation shown in FIG. 3B is a larger rotation (10-20 degrees around axis A) than that shown in FIG. 3A and may be sufficient to decouple the removable cap 19 and the housing 12, on one hand, and the sterile barrier 21 and the drug storage container 20, on the other hand. For example, in FIG. 3B the cap 19 has been rotated sufficiently such that the guard member 32 is visible between the cap 19 and the housing 12.

As discussed above, the device 10 requires a straight-pull force of approximately 10 to 15 Newtons to move the cap 19 from the storage position shown in FIG. 1 to the removed position shown in FIG. 4. In comparison, the rotational force required to move the cap from the storage position shown in FIG. 1 to the partially-removed position shown in FIG. 3 (e.g., “rotational-removal force”) is less than approximately 5 Newtons. As a another example, the rotational-removal force may be less than approximately 10 Newtons; less than approximately 8 Newtons; less than approximately 6 Newtons; less than approximately 4 Newtons; less than approximately 3 Newtons; less than approximately 2 Newtons; less than approximately 1.5 Newtons; less than approximately 1 Newtons; less than or equal to approximately 0.5 Newtons; approximately 0.5 Newtons. In the device 10 shown in FIGS. 1-5, removing the cap 19 requires approximately 0.5 Newtons of rotational-removal force.

The device may also include features that signal the camming function of the housing camming feature 12a and/or the camming function of the cap camming feature 19c. As an example, the housing camming feature 12a and/or the cap camming feature 19c may be visible to the user to signal the camming function of the camming features 12a, 19c. As a more specific example, some users may not readily appreciate that the removable cap 19 can/should be removed and/or that the removable cap 19 can/should be rotated. For example, some users may not be familiar with all the features or functions of drug delivery devices. As a more specific example, some users may not readily appreciate that the cap should be removed before use or how the cap can/should be removed. The device will typically include Instructions for Use (“IFUs”), but the visible camming features may reinforce the IFUs and/or give the user a visual signal on or near the end cap 19. As a result, the visibility of the camming features may improve ease of use, reduce user errors, improve user comfort with the device, reduce user complaints, and improve overall device compliance and user experience.

One or both of the camming features 12a, 19c may include additional aspects to improve the visibility thereof. For example, one or both of the camming features 12a, 19c may be brightly colored or highlighted or a different color than the surrounding features. As another example, the removable cap 19 may be translucent or transparent and the housing may have an opaque color. In such an embodiment, the guard member may be a bright color such as yellow or green to be visible through the translucent or transparent removable cap. As yet another example, one or both of the camming features 12a, 19c may include indicia encouraging or instructing the user to rotate the removable cap, such as arrows or other symbols, words, or other indicia. As another example, the housing 12 may be a light color such as white and the housing camming feature 12a may be a color (such as yellow, bright green, or bright orange) that stands-out from the rest of the housing 12 and the cap 19.

As an additional or alternative example, one or both of the camming features 12a, 19c may be defined by or positioned on the outer surface 25 of the drug delivery device 10 to signal the camming function of the features 12a, 19c. As another example, one or both of the camming features 12a, 19c may include indicia encouraging or instructing the user to rotate the removable cap, such as arrows or other symbols, words, or other indicia. As another example, one of the camming features may be distinguished by color scheme. Additionally or alternatively, the camming features 12a, 19c may be visually concentric and/or in close proximity to each other to help indicate the rotational camming function of the features.

The features that signal the camming function of the housing camming feature and the cap camming feature may be advantageous compared with or more desirable compared to camming features that are internal to the device or otherwise not visible to the user. For example, if the camming features are not visible then the user may not be able to easily view or appreciate the camming feature(s).

As discussed above, the design of the camming features 12a, 19c may affect various aspects of the cap removal, such as the rotational-removal force, the extent to which the features signal the camming function of the features 12a, 19c, the longitudinal distance that the cap travels when rotated (e.g., “cap lift”), and/or other aspects. For example, the friction coefficient and/or the cam angle of the camming features 12a, 19c may affect the rotational-removal force. As a more specific example, the friction coefficient for the camming features 12a, 19c shown in FIGS. 1-5 may be approximately 0.25. As another example, the friction coefficient for the camming features 12a, 19c may be between approximately 0.15 and 0.35; between approximately 0.15 and 0.5; between approximately 0.15 and 0.65; or between approximately 0.05 and 0.75. The friction coefficient may be affected by the materials comprising the camming features, the surface roughness, and/or the surface finish. It may be desirable to minimize the friction coefficient for the desired materials used in the device to minimize the rotational-removal force.

The cam angle 19m (e.g. the slope of a tangent line 19n of the cap camming feature 19c at the point of contact between the camming features 12a, 19c) shown in FIGS. 1-5 may be approximately 30 degrees. As another example, the cam angle 19m may be between approximately 25 and 35 degrees; between approximately 20 and 40 degrees; between approximately 15 and 45 degrees; between approximately 10 and 50 degrees; or between approximately 5 and 55 degrees. In general, a higher cam angle may cause a larger cap lift and a higher rotational-removal force. Conversely, a lower cam angle may cause a lower cap lift and a lower rotational-removal force. Therefore, it may be advantageous to select a cam angle that results in a desirable cap lift, cap rotation, and rotational-removal force. For example, a 90 degree rotation of the cap 19 shown in FIGS. 1-5 may cause a cap lift of approximately 5 mm and a rotational-removal force of 0.5 Nm. As another example, a 90 degree rotation of the cap 19 may cause a cap lift of approximately 4 to 6 mm and a rotational-removal force of between approximately 0.35 Nm to 0.65 Nm; a cap lift of approximately 5 to 7 mm and a rotational-removal force of between approximately 0.25 Nm to 0.75 Nm; a cap lift of approximately 3 to 8 mm and a rotational-removal force of between approximately 0.15 Nm to 0.85 Nm; or a cap lift of approximately 3 to 8 mm and a rotational-removal force of less than approximately 1 Nm.

The cam angle may vary at different points in the cap rotation. For example, the cam angle shown in FIG. 3B may be different than the cam angle shown in FIG. 3A. For example, the slope of a tangent line of the cap camming feature 19c may vary at different points along the cap camming feature 19c, such as if the cap camming feature 19c has a sinusoidal shape. The varying cam angle may be desirable to give the device a varying force profile. For example, it may be desirable to have a lower cam angle at the trough (e.g. low point) of the wave to minimize the initial rotational-removal force when the sterile barrier is still in tact. The cap camming feature 19c may then have a higher cam angle at a mid-point of the wave to provide a sufficient cap lift to decouple the cap from the housing. Conversely, it may be desirable to have a higher cam angle at the trough of the wave to initially and quickly decouple the cap from the housing to remove the sterile barrier. The cap camming feature 19c may then have a lower cam angle at a mid-point of the wave to have a low rotational-removal force and indicate to the user that the cap is decoupled from the housing.

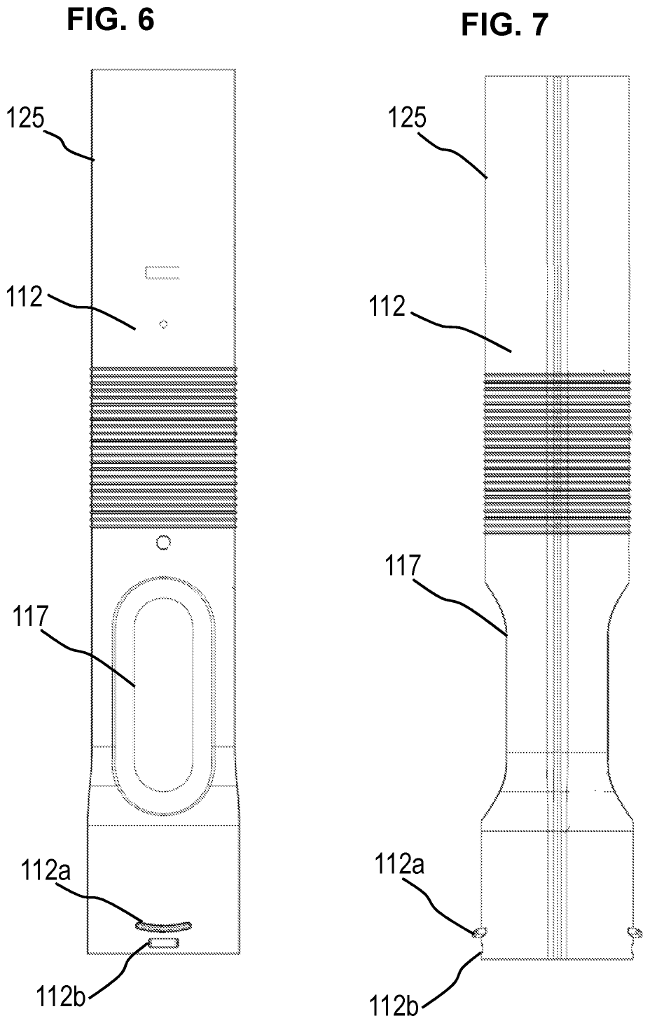

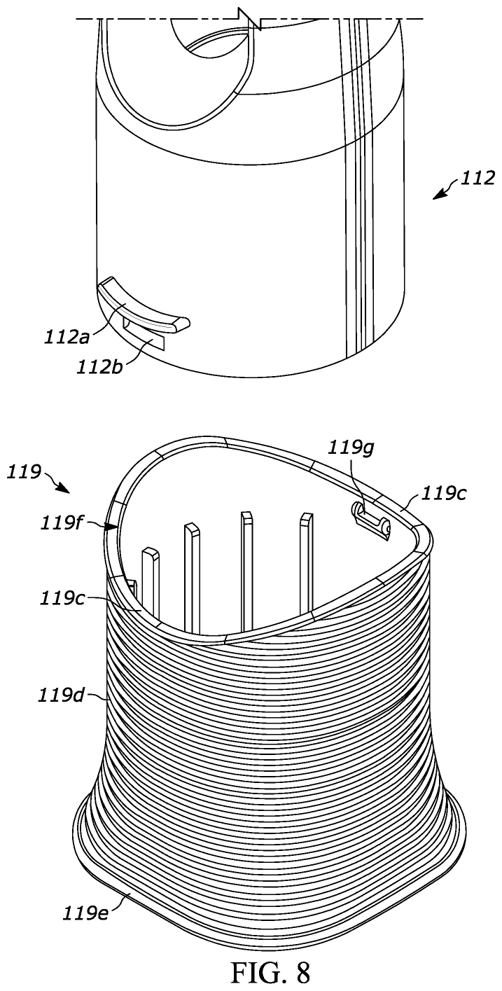

FIGS. 6-8 illustrate views of components of another exemplary drug delivery device in accordance with various embodiments. For example, FIGS. 6-7 show a housing 112 that may be coupled with other components to assembly a device such as that shown in FIGS. 1-5. The housing 112 includes many of the same features as the housing 12 shown in FIGS. 1-5, such as a viewing window 117, a housing outer surface 125, and a housing camming feature 112a. However, the housing camming feature 112a has a smaller curvature than the housing camming feature 12a shown in FIGS. 1-5. As discussed above, the camming features may have different shapes and sizes depending on the desired characteristics and attributes.

Additionally, the housing 112 includes a securing feature 112b to help secure a removable cap 119 to the housing 112. As a more specific example, the securing feature 112b includes an indentation or slot for receiving a securing tab formed in a cap. FIG. 8 shows the distal portion of the housing 112 positioned adjacent to the removable cap 119 configured to be removably coupled with the housing 112. For illustrative purposes, FIG. 8 does not show all of the components of a functioning device; rather it only shows the housing 112 and the removable cap 119. The cap 119 includes a securing tab 119g that is configured to fit within the indentation 112b to help secure the removable cap 119 to the housing 112. The securing features 112b, 119g may help to prevent inadvertent cap detachment/removal.

The securing feature 112b may be configured to engage the securing tab 119g to retain the removable cap 119 in the storage position. For example, the securing tab 119g may receive the securing feature 112b with a snap-fit connection that requires a baseline removal force to disengage the securing tab 119g from the securing feature 112b (or vice versa). As a more specific example, the securing tab 119g may have a size, shape, stiffness, and surface frictional characteristic that requires a removal force of 5 N to disengage the securing tab 119g from the securing feature 112b (or vice versa). Alternatively, the removal force may be between approximately 4 to 6 N; between approximately 3 to 7 N; between approximately 2 to 8 N; between approximately 1 to 9 N; between approximately 0 to 10 N; or another suitable value or range.

As an additional or alternative example, the securing feature 112b may retain the removable cap 119 in the storage position regardless of whether a drug storage container is located within the housing and/or whether the drug storage container is coupled with the housing in a position where the drug may be delivered. For example, it may be desirable for the removable cap 119 to be securable with the housing during an assembly stage when the drug storage container is not yet located within the housing or when the drug storage container is positioned within the housing but not yet in its final position with respect to the housing as shown in FIG. 5. To this end, the securing features 112b, 119g may be configured to secure the removable cap 119 with the housing 112 without the influence or assistance of other components such as the sterile barrier 21 or other components that couple the removable cap and the drug storage container (e.g., prior to final assembly of the device).

The securing features 112b, 119g may have alternate suitable configurations, such as a protrusion on the removable cap 119 and a receiving slot on the housing 112 or any other suitable features. The securing features 112b, 119g may also have any suitable shape, such as a curvature, a spiral shape, or a circular button-like shape. The shape, size, and other aspects of the securing features may facilitate removal of the cap in a particular direction or type of motion, such as rotational movement. For example, the securing features shown in FIGS. 6-8 are generally horizontal (e.g., generally perpendicular to the axis A) to promote decoupling when the removable cap 119 is rotated. The size and shape of the securing features 112b, 119g may also be designed in conjunction with the size and shape of the camming features 112a, 119c. For example, the camming features 112a, 119c may have a relatively flat bottom/trough portion to promote relative rotational motion between the housing and cap while minimizing relative translational motion until the securing features 112b, 119g are decoupled. In other words, the securing features 112b, 119g are rotationally separated/decoupled from each other before the cap is axially translated away from the housing. To this end, the securing features 112b, 119g are decoupled in a smooth movement rather than drag resistance and/or a jumping/snapping sensation for the user. Also, this configuration may have a relatively low or negligent effect on the rotational-removal force while providing a translational-removal force sufficient to secure the cap to the housing. In other words, the securing features 112b, 119g may prevent or minimize “stack” removal force.

The housing 112 and removable cap 119 include camming features to translate rotational motion into axial motion such that, upon rotational movement of the removable cap 119, the removable cap 119 is urged away from the housing 112, thereby facilitating and/or easing removal of the cap 119. For example, the housing 112 includes the housing camming feature 112a and a the removable cap 119 includes a cap camming feature 119c.

The cap camming feature 119c defines a wave shape, such as an arc-shaped surface. As a more specific example, the removable cap 119 shown in the figures includes a generally cylindrical body portion 119d and an end wall 119e that is generally perpendicular to the body portion 119d at the distal end of the cap 119. The body portion 119d defines a generally annular leading rim 119f at the proximal end of the cap 119. The leading rim 119f defines the wave shaped cap camming feature 119c. As an even more specific example, the leading rim 119f shown in the figures defines two wave shaped camming surfaces 119c.

The housing camming feature 112a defines a wave shape, such as an arc-shaped protrusion extending away from the outer surface of the housing 112. As a more specific example, the housing camming feature 112a is a protrusion having a shape that is not unlike a slightly-upturned mouth shape. As an even more specific example, the housing 112 shown in the figures defines two wave shaped camming features 112a.

When the removable cap 119 is in the storage position 119a, the cap camming features 119c engage or abut the housing camming features 112a. Additionally, the respective camming features 112a, 119c shown in the figures have matching or mirrored shapes such that the respective surfaces 112a, 119c slide smoothly/easily across each other. For example, when the removable cap 119 is rotated (either clockwise or counterclockwise) with respect to the housing 112, the housing camming features 112a, 119c rotate with respect to each other and urge the removable cap 119 away from the housing 112 along axis A. In other words, the camming features 112a, 119c translate rotational motion into axial motion to remove or assist with removal of the cap 119. As with the device shown in FIGS. 1-5, even a relatively small rotation of the removable cap 119 may facilitate and/or ease removal of the cap 119. The components shown in FIGS. 6-8 may result in a similar straight-pull force and rotational-removal force as those shown in FIGS. 1-5. As a more specific example, to remove the cap 119 from the housing 112 via axial force/movement only (straight-pull force), a user may be required to exert 45 Newtons or less; approximately 40 to 45 Newtons; approximately 35 to 40 Newtons; approximately 30 to 35 Newtons; approximately 25 to 30 Newtons; approximately 20 to 25 Newtons; approximately 15 to 20 Newtons; approximately 10 to 15 Newtons; approximately 5 to 10 Newtons; or less than approximately 5 Newtons. In the device shown in FIGS. 6-8, removing the cap 119 requires approximately 10 to 15 Newtons of straight-pull force. In comparison, the rotational force required to remove the cap from FIGS. 6-8 may be less than approximately 10 Newtons; less than approximately 8 Newtons; less than approximately 6 Newtons; less than approximately 4 Newtons; less than approximately 3 Newtons; less than approximately 2 Newtons; less than approximately 1.5 Newtons; less than approximately 1 Newtons; less than or equal to approximately 0.5 Newtons; approximately 0.5 Newtons. In the components shown in FIGS. 6-8, removing the cap 119 requires approximately 0.5 Newtons of rotational-removal force.

As with FIGS. 1-5, the device may also include features that signal the camming function of the housing camming feature 112a and/or the camming function of the cap camming feature 119c. For example, one or both of the camming features 112a, 119c may include additional aspects to improve the visibility thereof. As an additional or alternative example, one or both of the camming features 112a, 119c may be defined by or positioned on the outer surface 125 of the device to signal the camming function of the features 112a, 119c. As another example, one or both of the camming features 112a, 119c may include indicia encouraging or instructing the user to rotate the removable cap.

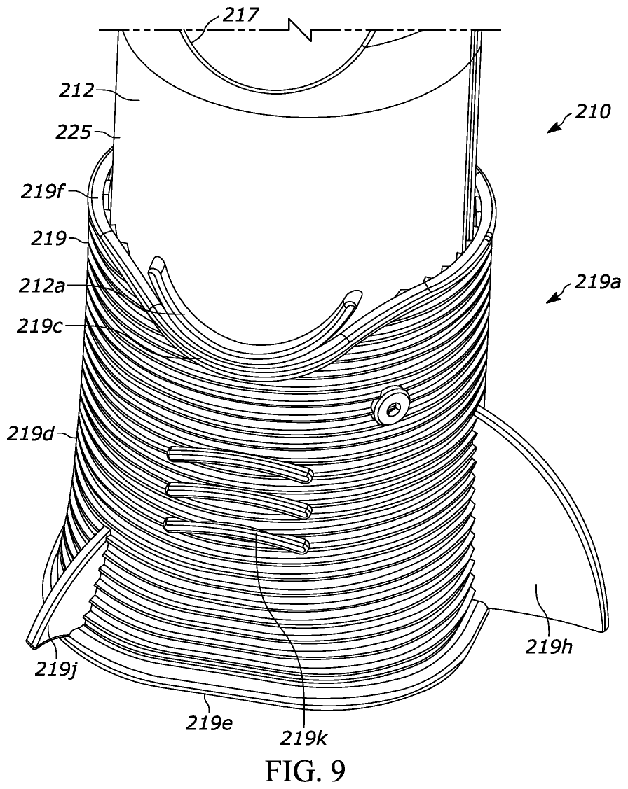

FIG. 9 illustrates views of components of another exemplary drug delivery device in accordance with various embodiments. For example, FIG. 9 shows a housing 212 and a removable cap 219 that may be coupled with each other to assembly a device such as that shown in FIGS. 1-5. The housing 212 includes many of the same features as the housing 12 shown in FIGS. 1-5, such as a viewing window 217, a housing outer surface 225, and a housing camming feature 212a.

The housing 212 and end cap 219 include camming features to translate rotational motion into axial motion such that, upon rotational movement of the removable cap 219, the removable cap 219 is urged away from the housing 212, thereby facilitating and/or easing removal of the cap 219. For example, the housing 212 includes the housing camming feature 212a and a the removable cap 219 includes a cap camming feature 219c.

The cap camming feature 219c defines a wave shape, such as an arc-shaped surface. As a more specific example, the removable cap 219 shown in the figures includes a generally cylindrical body portion 219d and an end wall 219e that is generally perpendicular to the body portion 219d at the distal end of the cap 219. The body portion 219d defines a generally annular leading rim 219f at the proximal end of the cap 219. The leading rim 219f defines the wave shaped cap camming feature 219c. As an even more specific example, the leading rim 219f shown in the figures defines two wave shaped camming surfaces 219c.

The housing camming feature 212a defines a wave shape, such as an arc-shaped protrusion extending away from the outer surface of the housing 212. As a more specific example, the housing camming feature 212a is a protrusion having a shape that is not unlike a smile or a crescent moon shape. As an even more specific example, the housing 212 shown in the figures defines two wave shaped camming features 212a.

When the removable cap 219 is in the storage position 219a, the cap camming features 219c engage or abut the housing camming features 212a. Additionally, the respective camming features 212a, 219c shown in the figures have matching or mirrored shapes such that the respective surfaces 212a, 219c slide smoothly/easily across each other. For example, when the removable cap 219 is rotated (either clockwise or counterclockwise) with respect to the housing 212, the housing camming features 212a, 219c rotate with respect to each other and urge the removable cap 219 away from the housing 212 along axis A. In other words, the camming features 212a, 219c translate rotational motion into axial motion to remove or assist with removal of the cap 219. As with the device shown in FIGS. 1-5, even a relatively small rotation of the removable cap 219 may facilitate and/or ease removal of the cap 219. The components shown in FIG. 9 may result in a similar straight-pull force and rotational-removal force as those shown in FIGS. 1-5. As a more specific example, to remove the cap 219 from the housing 212 via axial force/movement only (straight-pull force), a user may be required to exert 45 Newtons or less; approximately 40 to 45 Newtons; approximately 35 to 40 Newtons; approximately 30 to 35 Newtons; approximately 25 to 30 Newtons; approximately 20 to 25 Newtons; approximately 15 to 20 Newtons; approximately 10 to 15 Newtons; approximately 5 to 10 Newtons; or less than approximately 5 Newtons. In the device shown in FIG. 9, removing the cap 219 requires approximately 10 to 15 Newtons of straight-pull force. In comparison, the rotational force required to remove the cap from FIG. 9 may be less than approximately 10 Newtons; less than approximately 8 Newtons; less than approximately 6 Newtons; less than approximately 4 Newtons; less than approximately 3 Newtons; less than approximately 2 Newtons; less than approximately 1.5 Newtons; less than approximately 1 Newtons; less than or equal to approximately 0.5 Newtons; approximately 0.5 Newtons. In the components shown in FIG. 9, removing the cap 219 requires approximately 0.5 Newtons of rotational-removal force.

The cap 219 shown in FIG. 9 may also include rotation-assistance features, such as fins 219h, 219j that increase user gripability and/or increase the torque that a user is able to exert on the cap 219. The fins 219h, 219j shown in FIG. 9 may also be shaped, sized, and spaced apart such as to ergonomically fit a users hand or fingers. For example, the fin 219j may fit a users thumb and the fin 219h may fit the user's index and/or middle finger. The fins may also have a size suitable for exerting the desirable amount of torque on the cap 219, such as a fin thickness sufficient to prevent fin breakage or warping and a fin height sufficient to generate a desirable moment arm around the longitudinal axis of the cap.

The cap 219 shown in FIG. 9 may also include a grip-assistance features, such as ribs 219k that increase user gripability for a user to exert a longitudinal force on the cap 219. The ribs 219k shown in FIG. 9 may also be shaped, sized, and spaced apart such as to ergonomically fit a user's hand or fingers. For example, the ribs 219k may fit a user's thumb and index finger. The ribs 219k may also have a size suitable for exerting a desirable amount of axial force on the cap 219, such as a rib thickness sufficient to prevent fin breakage or warping and a rib orientation (slightly concave) to signal to the user that the cap should be pulled downward in the direction of the ends of the ribs.

As with FIGS. 1-8, the device may also include features that signal the camming function of the housing camming feature 212a and/or the camming function of the cap camming feature 219c. For example, one or both of the camming features 212a, 219c may include additional aspects to improve the visibility thereof. As an additional or alternative example, one or both of the camming features 212a, 219c may be defined by or positioned on the outer surface 225 of the device to signal the camming function of the features 212a, 219c. As another example, one or both of the camming features 212a, 219c may include indicia encouraging or instructing the user to rotate the removable cap.

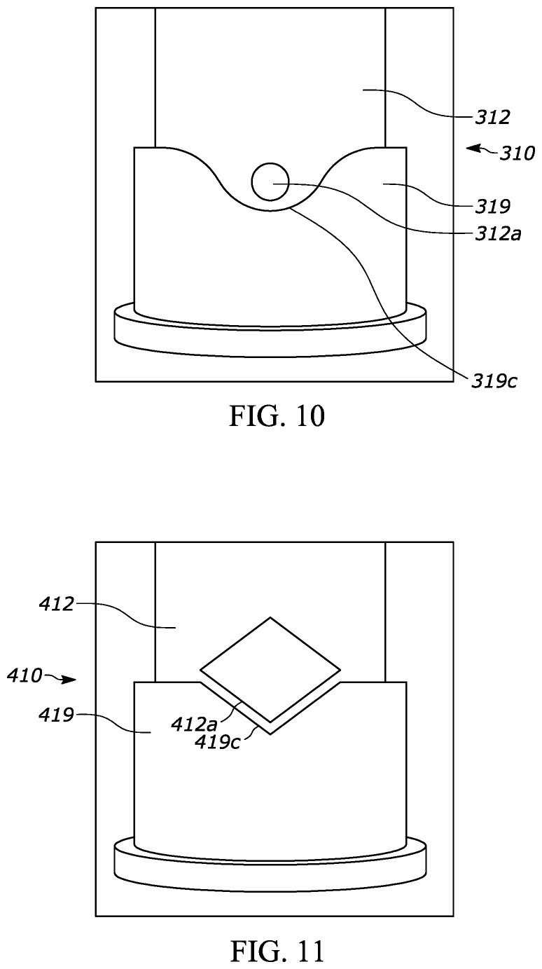

FIG. 10 illustrates a view of components of an additional exemplary drug delivery device in accordance with various embodiments. For example, FIG. 10 shows the distal portion of a device 310 having a housing 312 and an end cap 319 that may be coupled with each other to assembly a device such as that shown in FIGS. 1-5. The device 310 in FIG. 10 includes many of the same features as the device shown in FIGS. 1-5, such as a viewing window, a housing outer surface, and a housing camming feature 312a. The end cap 319c includes camming features to translate rotational motion into axial motion such that, upon rotational movement of the removable cap 319, the removable cap 319 is urged away from the housing 312, thereby facilitating and/or easing removal of the cap 319. For example, the housing 312 includes the housing camming feature 312a and the removable cap 319 includes cap camming feature 319c. The cap camming feature shown in FIG. 10 is generally wave shaped and the housing camming feature 312a is a circular or rounded protrusion. When the removable cap 319 is in the storage position 319a, the cap camming features 319c engage or abut the housing camming features 312a. Additionally, the respective camming features 312a, 319c shown in the figures have matching or mirrored shapes such that the respective surfaces 312a, 319c slide smoothly/easily across each other. For example, when the removable cap 319 is rotated (either clockwise or counterclockwise) with respect to the housing 312, the housing camming features 312a, 319c rotate with respect to each other and urge the removable cap 319 away from the housing 312 along axis A. In other words, the camming features 312a, 319c translate rotational motion into axial motion to remove or assist with removal of the cap 319.

FIG. 11 illustrates a view of components of yet another exemplary drug delivery device in accordance with various embodiments. For example, FIG. 11 shows the distal portion of a device 410 having a housing 412 and an end cap 419 that may be coupled with each other to assembly a device such as that shown in FIGS. 1-5. The device 410 in FIG. 11 includes many of the same features as the device shown in FIGS. 1-5, such as a viewing window, a housing outer surface, and a housing camming feature 412a. The end cap 419c includes camming features to translate rotational motion into axial motion such that, upon rotational movement of the removable cap 419, the removable cap 419 is urged away from the housing 412, thereby facilitating and/or easing removal of the cap 419. For example, the housing 412 includes the housing camming feature 412a and the removable cap 419 includes cap camming feature 419c. The cap camming feature shown in FIG. 11 is generally triangular-shaped and the housing camming feature 412a is a triangle-shaped or diamond-shaped protrusion. When the removable cap 419 is in the storage position 419a, the cap camming features 419c engage or abut the housing camming features 412a. Additionally, the respective camming features 412a, 419c shown in the figures have matching or mirrored shapes such that the respective surfaces 412a, 419c slide smoothly/easily across each other. For example, when the removable cap 419 is rotated (either clockwise or counterclockwise) with respect to the housing 412, the housing camming features 412a, 419c rotate with respect to each other and urge the removable cap 419 away from the housing 412 along axis A. In other words, the camming features 412a, 419c translate rotational motion into axial motion to remove or assist with removal of the cap 419.

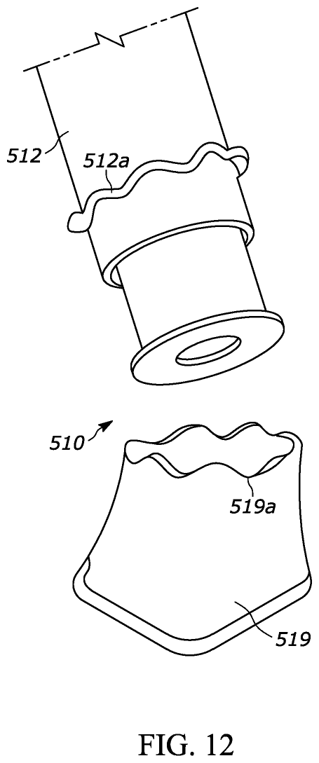

FIG. 12 illustrates a view of components of yet another exemplary drug delivery device in accordance with various embodiments. For example, FIG. 12 shows the distal portion of a device 510 having a housing 512 and an end cap 519 that may be coupled with each other to assembly a device such as that shown in FIGS. 1-5. The device 510 in FIG. 12 includes many of the same features as the device shown in FIGS. 1-5, such as a viewing window, a housing outer surface, and a housing camming feature 512a. The end cap 519c includes camming features to translate rotational motion into axial motion such that, upon rotational movement of the removable cap 519, the removable cap 519 is urged away from the housing 512, thereby facilitating and/or easing removal of the cap 519. For example, the housing 512 includes the housing camming feature 512a and the removable cap 519 includes cap camming feature 519c. The cap camming feature shown in FIG. 12 is generally wave-shaped, namely a continuous or semi-continuous wave shape extending around the circumference of the cap. The housing camming feature 512a is generally wave-shaped, namely a continuous or semi-continuous wave shape extending around the circumference of the cap. When the removable cap 519 is in the storage position 519a, the cap camming features 519c engage or abut the housing camming features 512a. Additionally, the respective camming features 512a, 519c shown in the figures have matching or mirrored shapes such that the respective surfaces 512a, 519c slide smoothly/easily across each other. For example, when the removable cap 519 is rotated (either clockwise or counterclockwise) with respect to the housing 512, the housing camming features 512a, 519c rotate with respect to each other and urge the removable cap 519 away from the housing 512 along axis A. In other words, the camming features 512a, 519c translate rotational motion into axial motion to remove or assist with removal of the cap 519.

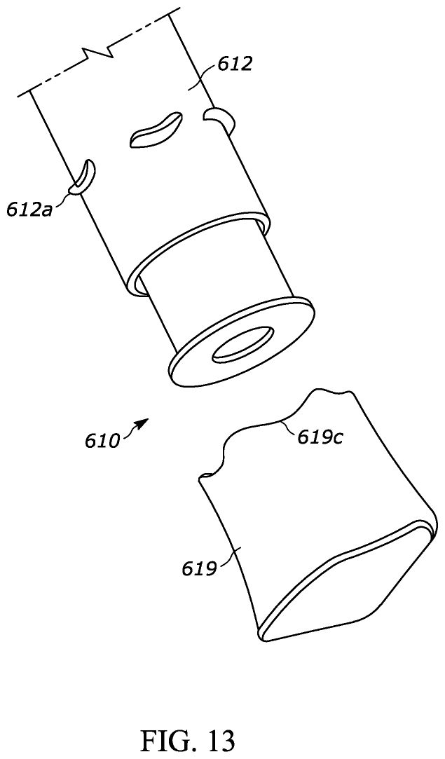

FIG. 13 illustrates a view of components of yet another exemplary drug delivery device in accordance with various embodiments. For example, FIG. 13 shows the distal portion of a device 610 having a housing 612 and an end cap 619 that may be coupled with each other to assembly a device such as that shown in FIGS. 1-5. The device 610 in FIG. 13 includes many of the same features as the device shown in FIGS. 1-5, such as a viewing window, a housing outer surface, and a housing camming feature 612a. The end cap 619c includes camming features to translate rotational motion into axial motion such that, upon rotational movement of the removable cap 619, the removable cap 619 is urged away from the housing 612, thereby facilitating and/or easing removal of the cap 619. For example, the housing 612 includes the housing camming feature 612a and the removable cap 619 includes cap camming feature 619c. The cap camming feature shown in FIG. 13 is generally wave-shaped, namely a continuous or semi-continuous wave shape extending around the circumference of the cap. The housing camming feature 612a is a plurality of protrusions having a curvature or a wave shape. When the removable cap 619 is in the storage position 619a, the cap camming features 619c engage or abut the housing camming features 612a. Additionally, the respective camming features 612a, 619c shown in the figures have matching or mirrored shapes such that the respective surfaces 612a, 619c slide smoothly/easily across each other. For example, when the removable cap 619 is rotated (either clockwise or counterclockwise) with respect to the housing 612, the housing camming features 612a, 619c rotate with respect to each other and urge the removable cap 619 away from the housing 612 along axis A. In other words, the camming features 612a, 619c translate rotational motion into axial motion to remove or assist with removal of the cap 619.

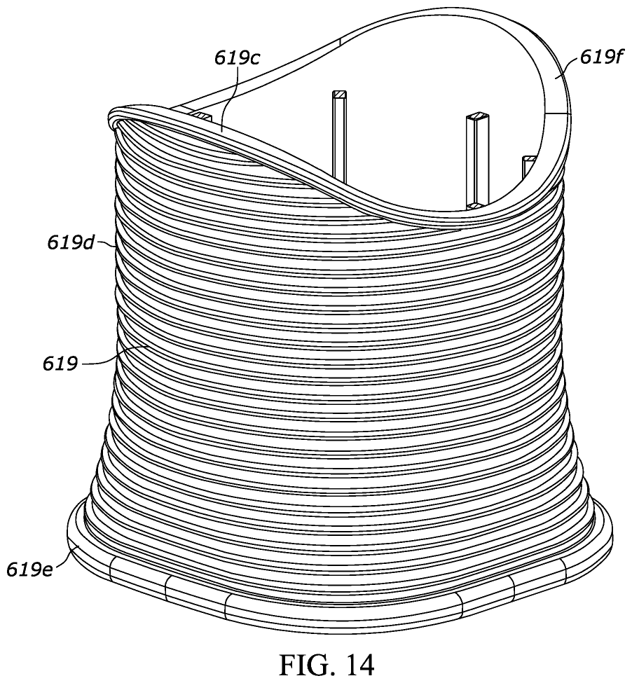

FIG. 14 illustrates a view of a component, namely an end cap, of yet another exemplary drug delivery device in accordance with various embodiments. For example, the end cap 619 includes a cap camming feature 619c that defines a wave shape, such as an arc-shaped surface. As a more specific example, the removable cap 619 shown in the figures includes a generally cylindrical body portion 619d and an end wall 619e that is generally perpendicular to the body portion 619d at the distal end of the cap 619. As an even more specific example, the leading rim 619f shown in the figures defines a continuous wave shaped camming surfaces 619c, such as a sinusoidal wave shaped camming surface 619c.

From the foregoing, it can be seen that the present disclosure advantageously provides a streamlined design for a drug delivery device having automated features. Various mechanisms and components of the drug delivery device may interact with each other in synergistic ways so as to limit the number of moving parts required by the drug delivery device, thereby improving the reliability of the drug delivery device and saving costs, as well as providing other benefits and advantages.

As will be recognized, the devices and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“ID-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-?4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNF? monoclonal antibody); Reopro® (abciximab, anti-GP IIb/IIIa receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-?4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2R? mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNF? mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-?5?1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFN? mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG? mAb (MDX-1307); anti-mesothelin dsFv-PE38 conjugate (CAT-5001); anti-PD1mAb (MDX-1106 (ONO-4538)); anti-PDGFR? antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF ? monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing defining a longitudinal axis and a camming feature, the housing having an opening;
a drug storage container including a delivery member having an insertion end configured to extend at least partially through the opening during a delivery state; and
a removable cap defining a cap camming feature and configured to be removably coupled with the housing such that the removable cap has a storage position where the removable cap is coupled with the housing and at least partially covering the opening and a removed position where the removable cap is not coupled with the housing;
wherein the cap camming feature and the housing camming feature are configured to translate rotational motion into axial motion such that, upon rotational movement of the removable cap, the cap camming feature and/or the housing camming feature urge the removable cap along the longitudinal axis; and
wherein the housing camming feature and the cap camming feature are each visible to a user of the drug delivery device to signal the camming function of the housing camming feature and/or the cap camming feature.

2. The drug delivery device of claim 1, wherein the cap camming feature defines a wave shape.

3. The drug delivery device of claim 2, wherein the wave shape is a generally sinusoidal shape.

4. The drug delivery device of claim 3, wherein the removable cap includes a generally cylindrical body portion defining an annular leading rim and an end wall generally perpendicular to the body portion and wherein the annular leading rim defines the wave shape.

5. The drug delivery device of claim 2, wherein the housing defines a generally cylindrical outer surface and the housing camming feature includes a protrusion extending away from a generally cylindrical outer surface.

6. The drug delivery device of claim 5, wherein the protrusion is aligned with the wave shape of the cap camming feature when the removable cap is in the storage position.

7. The drug delivery device of claim 6, wherein the protrusion abuts the wave shape of the cap camming feature when the removable cap is in the storage position.

8. The drug delivery device of claim 7, wherein the protrusion defines a wave surface corresponding to the wave shape of the cap camming feature.

9. The drug delivery device of claim 6, wherein the housing includes two protrusions each extending away from a generally cylindrical outer surface.

10. The drug delivery device of claim 1, wherein the housing defines a securing feature configured to engage the removable cap and retain the removable cap in the storage position.

11. The drug delivery device of claim 10, wherein the securing feature is configured to retain the removable cap in the storage position regardless of whether the drug storage container is coupled with the housing.

12. The drug delivery device of claim 1, wherein the housing camming feature and the cap camming feature are each defined by or positioned on an outer surface of the drug delivery device.

13. The drug delivery device of claim 1, wherein at least one of the housing camming feature and/or the cap camming feature includes an aspect to improve the visibility thereof, wherein optionally, the aspect to improve the visibility includes a bright color.

14. (canceled)

15. The drug delivery device of claim 1, wherein the cap includes at least one rotation-assistance feature, wherein optionally, the at least one rotation-assistance feature includes a fin.

16. (canceled)

17. A drug delivery device comprising:
a housing defining a longitudinal axis and a camming feature, the housing having an opening;
a drug storage container including a delivery member having an insertion end configured to extend at least partially through the opening during a delivery state; and
a removable cap defining a cap camming feature and configured to be removably coupled with the housing such that the removable cap has a storage position where the removable cap is coupled with the housing and at least partially covering the opening and a removed position where the removable cap is not coupled with the housing;
wherein the cap camming feature and the housing camming feature are configured to translate rotational motion into axial motion such that, upon rotational movement of the removable cap, the cap camming feature and/or the housing camming feature urge the removable cap along the longitudinal axis; and
wherein the housing camming feature and the cap camming feature are each defined by or positioned on an outer surface of the drug delivery device to signal the camming function of the housing camming feature and/or the cap camming feature.

18. The drug delivery device of claim 17, wherein the cap camming feature defines a wave shape; and
wherein the removable cap includes a generally cylindrical body portion defining an annular leading rim and an end wall generally perpendicular to the body portion and wherein the annular leading rim defines the wave shape.

19. The drug delivery device of claim 18, wherein the housing defines a generally cylindrical outer surface and the housing camming feature includes a protrusion extending away from a generally cylindrical outer surface.

20. The drug delivery device of claim 19, wherein the protrusion is aligned with the wave shape of the cap camming feature when the removable cap is in the storage position.

21. The drug delivery device of claim 17, wherein at least one of the housing camming feature and/or the cap camming feature includes an aspect to improve the visibility thereof.

22. The drug delivery device of claim 17, wherein the cap includes at least one rotation-assistance feature.
